(19) United States
(12) Patent Application Publication
(10) Pub. No.: US 2013/0084544 A1
(43) Pub. Date: Apr. 4, 2013

(54) Cavitation Medication Delivery System

(71) Applicant: BIOLASE, INC., Irvine, CA (US)

(72) Inventors: Dmitri Boutoussov, Dana Point, CA (US); Michael Radicone, Huntington Station, NY (US); Vladimir Lemberg, Santa Clara, CA (US); Vladimir Netchitailo, Livermore, CA (US)

(73) Assignee: BIOLASE, INC., Irvine, CA (US)

(21) Appl. No.: 13/632,788

(22) Filed: Oct. 1, 2012

Related U.S. Application Data
(60) Provisional application No. 61/541,029, filed on Sep. 29, 2011.

(51) Int. Cl. A61M 37/00; A61C 19/06

(52) U.S. Cl. 433/215; 604/20

(57) ABSTRACT

Systems and methods for delivering a substance to a target region in vapor form are provided. A fluid is placed within an interaction zone, where the interaction zone is a volume that extends into the target region or that is adjacent to the target region. A fiber optic tip is placed within the interaction zone. The fiber optic tip contains the substance that is transparent to a first wavelength of energy and that substantially absorbs a second wavelength of energy. A vapor bubble is created within the interaction zone by exposing the fluid to electromagnetic radiation at the first wavelength, where the radiation at the first wavelength is substantially absorbed by the fluid. The substance is released in vapor form into the vapor bubble by exposing the substance to electromagnetic radiation at the second wavelength. The fiber optic tip emits the radiation at the first and second wavelengths.

### CROSS-REFERENCE TO RELATED APPLICATIONS

This application claims priority to U.S. Provisional Patent Application No. 61/541,029, filed Sep. 29, 2011, entitled “Cavitation Medication Delivery System,” which is herein incorporated by reference in its entirety.

### TECHNICAL FIELD

The technology described herein relates generally to the delivery of a substance to a target region and more particularly to the use of electromagnetic radiation emitting devices for delivering a substance to a target region via a vapor bubble.

### BACKGROUND

A primary cause of infection, disease, and death in humans is inadequate bacteria control. Thus, killing or removing bacteria from various systems of the human body is an important part of many medical and dental procedures. For example, during a root canal procedure, the root canal is disinfected by mechanical debridement of the canal wall and an application of an antiseptic substance within the canal to kill remaining bacteria. However, dental technology has found it difficult to completely eradicate all bacteria during a root canal procedure. In particular, the structural anatomy of the tooth makes it difficult to eliminate all bacteria because the root canal includes irregular canals and microscopic tubules where bacteria can lodge and fester. Bacteria control in other medical and dental procedures has proven equally difficult, and the failure to control bacteria during these procedures can lead to a variety of health and medical problems (e.g., presence of bacteria in the bloodstream, infection of organs including the heart, lung, kidneys, and spleen).

### SUMMARY

Systems and methods are provided for delivering a substance to a target region in a vapor form. In a method for delivering a substance to a target region in a vapor form, a fluid is placed within an interaction zone, where the interaction zone is a volume that extends into the target region or that is adjacent to the target region. An electromagnetic radiation emitting fiber optic tip is positioned within the interaction zone. The fiber optic tip contains the substance that is transparent to a first wavelength of energy and that substantially absorbs a second wavelength of energy. A vapor bubble is created within the interaction zone by exposing the fluid to electromagnetic radiation at the first wavelength, where the electromagnetic radiation at the first wavelength is substantially absorbed by the fluid in the interaction zone. The substance is released in a vapor form into the vapor bubble by exposing the substance to electromagnetic radiation at the second wavelength. The electromagnetic radiation at the first and second wavelengths are emitted by the fiber optic tip.

A system for delivering a substance to a target region in a vapor form includes a fluid, where the fluid is located within an interaction zone that is a volume extending into the target region or adjacent to the target region. The system also includes an electromagnetic radiation emitting fiber optic tip. The fiber optic tip is positioned within the interaction zone and contains the substance that is transparent to a first wavelength of energy and that substantially absorbs a second wavelength of energy. The system further includes an electromagnetic energy source. The electromagnetic energy source is configured to generate electromagnetic radiation at the first and second wavelengths for emission by the fiber optic tip. The emitted electromagnetic radiation at the first wavelength is substantially absorbed by the fluid and is configured to create a vapor bubble within the fluid. The emitted electromagnetic radiation at the second wavelength is configured to release the substance in a vapor form into the vapor bubble.

In another method for delivering a substance to a target region in a vapor form, a fluid is placed within an interaction zone. The interaction zone is a volume that extends into the target region or that is adjacent to the target region. An electromagnetic radiation emitting element is positioned within the interaction zone, where the element contains the substance that is transparent to a particular wavelength of energy. A vapor bubble is created within the fluid by exposing the fluid to electromagnetic radiation at the particular wavelength. The electromagnetic radiation at the particular wavelength is emitted by the electromagnetic radiation emitting element and is substantially absorbed by the fluid in the interaction zone. During the creation of the vapor bubble, the substance is released into the vapor bubble.

### BRIEF DESCRIPTION OF THE FIGURES

FIGS. 1A, 1B, 1C, and 1D depict an example system for delivering a substance to a target region in a vapor form.

FIG. 2 depicts a block diagram of an example system utilizing a dual-wavelength electromagnetic energy source and a multi-mode fiber optic cable to deliver a substance to a target region in a vapor form.

FIG. 3 depicts example timing diagrams illustrating aspects of a method for delivering a substance to a target region in a vapor form.

FIG. 4 depicts fiber optic cables inserted into root canals of a tooth for intra-canal disinfection, cleaning, and/or medication delivery.

FIG. 5 illustrates an example system for delivering a medication or cleaning agent to a target area via a plurality of vapor bubbles carrying the medication or the cleaning agent in a vapor form.

FIGS. 6A and 6B depict example systems that utilize a spraying technique to disperse medication into a vapor bubble for delivery to a target region.

FIG. 7 depicts a block diagram of an example system utilizing an electromagnetic energy source with a plurality of laser sources to deliver a medicine to a target region in a vapor form.

FIG. 8 is a flowchart illustrating an example method for delivering a substance to a target region in a vapor form.

### DETAILED DESCRIPTION

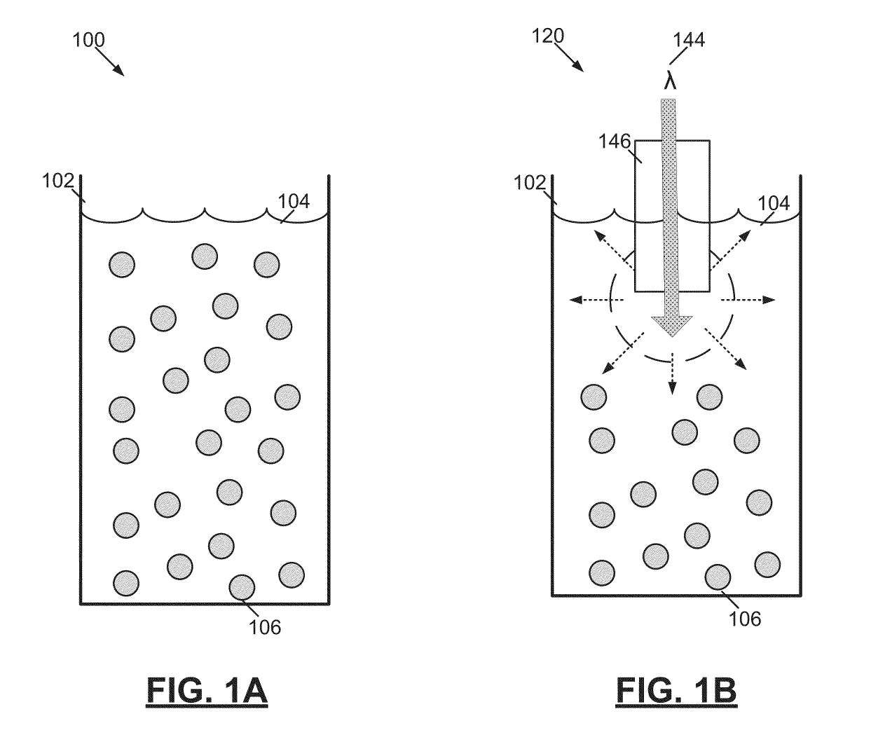

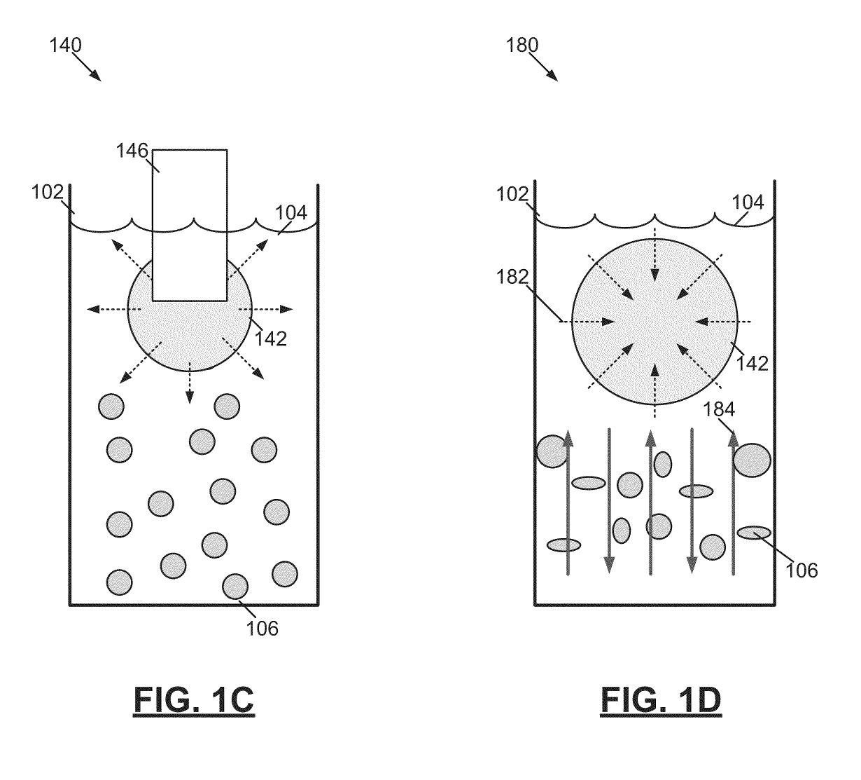

FIGS. 1A, 1B, 1C, and 1D depict an example system for delivering a substance 108 to a target region 102 in a vapor form. FIG. 1A depicts the example system during a first period of time 100. In FIG. 1A, a fluid 104 is placed within the target region 102. The fluid 102 may be, for example, a water-based solution or a saline solution. The target region 102 is a cavity, canal, passage, opening, or surface to which it is desired that the substance 108 be delivered (e.g., a root canal to which it is desired that iodine be delivered to kill bacteria). During the first period of time 100, in addition to the fluid 104 being placed in the target region 102, a fiber optic tip 106 is also positioned within the target region 102. The fiber optic tip 106 is an electromagnetic radiation emitting fiber optic tip and is connected via a multi-mode fiber optic cable to an electromagnetic energy source. The electromagnetic energy source generates electromagnetic radiation that is routed along the multi-mode fiber optic cable and emitted by the fiber optic tip 106. As illustrated in FIG. 1A, the fiber optic tip 106 is coated in the substance 108 to be delivered to the target region 102. The fiber optic tip 106 may be coated in any adequate manner (e.g., via dip-coating and/or various deposition techniques including sputtering and evaporation). The substance 108 coats the fiber optic tip 106 such that electromagnetic radiation of certain wavelengths emitted by the fiber optic tip 106 interacts with the substance 108 as it is emitted from the tip 106.

The fiber optic tip 106 may be of a variety of different shapes (e.g., conical, angled, beveled, double-beveled), sizes, designs (e.g., side-firing, forward-firing), and materials (e.g., glass, sapphire, quartz, hollow waveguide, liquid core, quartz silica, germanium oxide). In one example, the fiber optic tip 106 is made of glass with a diameter of 400 μm, and the substance 108 coating the fiber optic tip 106 is iodine having a coating thickness of 1-2 μm. Further, although the system of FIGS. 1A, 1B, 1C, and 1D illustrates the use of the fiber optic tip 106 as the light emitting element of the system, in other examples, various waveguides, light emitting elements (e.g., light emitting nanoparticles and nanostructures, quantum dots), and/or devices including mirrors, lenses, and other optical components may be used in place of the fiber optic tip 106 for light emission.

During a second period of time 140, a vapor bubble 142 is created within the target region 102. The vapor bubble 142 is created by exposing the fluid 104 to electromagnetic radiation at a first wavelength 144. The exposing of the fluid 104 is accomplished by focusing or placing a peak concentration of the electromagnetic radiation at the first wavelength 144 on the fluid 104 using the fiber optic tip 106. The first wavelength 144 is selected to be substantially absorbed by the fluid 104 and transparent to the substance 108. Thus, the electromagnetic radiation at the first wavelength 144 is generated by the electromagnetic energy source, routed to the fiber optic tip 106 via the multi-mode fiber optic cable, and emitted via the fiber optic tip 106 into the fluid 104. The electromagnetic radiation at the first wavelength 144 passes through the substance 108 coating the fiber optic tip 106 in a relatively unimpeded manner because of the transparency of the substance 108 to the first wavelength 144. Due to the high absorption of the first wavelength 144 in the fluid 104, the vapor bubble 142 forms near the end of the fiber optic tip 106.

As noted above, the fluid 104 substantially absorbs electromagnetic radiation at the first wavelength 144. In FIG. 1B, the fluid 104 is a water-based solution, and the first wavelength 144 is within the range of 2.6 μm-3.1 μm, which is substantially absorbed by water. In one example, the electromagnetic radiation at the first wavelength 144 is delivered to the fluid 104 as a pulse of light, rather than as a continuous, steady-state beam of light. In another example, the electromagnetic radiation at the first wavelength 144 has a wavelength of 2.79 μm, a pulse width of 50 μs, a pulse energy of 20 mJ, and a peak power of 400 W.

During a third period of time 180, the substance 108 is released in a vapor form 182 into the vapor bubble 142. The substance 108 is released in vapor form 182 by exposing the substance 108 to electromagnetic radiation at a second wavelength 184. The second wavelength 184 is selected to be substantially absorbed by the substance 108. The electromagnetic radiation at the second wavelength 184 is generated by the electromagnetic energy source, routed to the fiber optic tip 106 via the multi-mode fiber optic cable, emitted via the fiber optic tip 106, and absorbed within the substance 108 coating the fiber optic tip 106. The power of any electromagnetic radiation at the second wavelength 184 that reaches the fluid 104 is highly attenuated due to the high absorption of the second wavelength 184 in the substance 108. The absorption of the electromagnetic radiation at the second wavelength 184 by the substance 108 causes the substance 108 to evaporate into the vapor bubble 142. Although FIGS. 1B and 1C depict the electromagnetic radiation at the first and the second wavelengths 144, 184 as being emitted independently of each other, in some systems, the first and second wavelengths 144, 184 are pulses of light launched at substantially similar times. In these systems, the substance 108 is released in vapor form 182 into the vapor bubble 142 during a period of time in which the vapor bubble 142 is being created. The vapor bubble 142 containing the substance 108 in vapor form 182 is used to deliver the substance 108 to various parts of the target region 102.

In the system illustrated in FIG. 1C, the second wavelength 184 is configured to match an absorption peak of the substance 108 and may be within a range of 350 nm-2500 nm, which includes electromagnetic radiation within the ultraviolet, visible, and near-infrared regions of the electromagnetic spectrum. In an example system, the electromagnetic radiation at the second wavelength 184 is delivered to the substance 108 as a pulse of light, where the electromagnetic radiation at the second wavelength 184 has a wavelength of 940 nm, a pulse width of 1 ms, a pulse energy of 1 mJ, and a peak power of 1 W.

In the system 190 illustrated in FIG. 1D, the tip 106 has five open channels 192, which are used to incorporate the substance 108 into the vapor bubble 142. The substance 108 is not coated over the end of the tip 106, as in the preceding figures. The substance 108 can thus be in the form of the coating over the end of fiber optic tip 106, or the substance 108 can be impregnated into pores of the tip 106 itself.

Although the vapor bubble 142 is described herein primarily as a means of delivering the substance 108 in vapor form 182 to the target region 102, in some systems, the vapor bubble 142 may itself play a role in achieving disinfection, cleaning, and/or other functions in the target region 102. As described above, the vapor bubble 142 is created by exposing the fluid 104 to the electromagnetic radiation at the first wavelength 144. In an example system, an initial pulse of radiation operates to generate the vapor bubble 142. Following this initial pulse, additional radiation pulses expand the vapor bubble 142 until the pressure on the outside of the vapor bubble 142 reaches a limit, and the bubble collapses, creating shock waves in the fluid 104. The shock waves can clean and/or disrupt (e.g., remove) substances within the target region 102 (e.g., remove and/or kill bacteria within the target region 102). In other systems, the vapor bubble 142 may be engineered to explode rapidly, which can be used to impart strong, concentrated forces on the target region 102 and/or particles within the target region 102.

The target region 102 may be of a small size (e.g., on the order of the size of the fiber optic tip 106) and may be a cavity, canal, passage, opening, or surface of the human body (e.g., a root canal passage, tubule of a tooth, tooth cavity, blood vessel). Thus, the system of FIGS. 1A, 1B, 1C, and 1D for delivering the substance 108 to the target region 102 may be employed in the context of a variety of medical or dental procedures (e.g., treating tissue, removing deposits and stains from surfaces, removing or killing bacteria). For example, the system of FIGS. 1A, 1B, 1C, and 1D may be used as part of a root canal treatment procedure, where the substance 108 is a medicine, cleaning agent, biologically-active particle, antiseptic, or antibiotic, and the target region 102 is a portion of a root canal. The substance 108 is configured to clean, remove bacteria, kill bacteria, disinfect, and/or apply a medical treatment to the root canal.

Non-dental applications of the system of FIGS. 1A, 1B, 1C, and 1D include procedures within a human body passage, such as a vessel (e.g., blood vessel) or an opening, cavity, or lumen within hard or soft tissue (e.g., treatment of occluded arteries or necrotic bone). Another use of the system of FIGS. 1A, 1B, 1C, and 1D is in the treatment of a surface condition of the skin (e.g., skin having an acne condition), where the substance 108 used to treat the surface condition includes an antibacterial agent such as minocycline hydrochloride. Substances that may be delivered to the target region 102 include medications, such as antibiotics, steroids, anesthetics, anti-inflammatory treatments, antiseptics, disinfectants, adrenaline, epinephrine, astringents, vitamins, herbs, and minerals. In one particular system, the substance 108 to be delivered to the target region 102 is iodine, and the iodine is configured to kill bacteria within the fluid 104 and/or on walls of the target region 102.

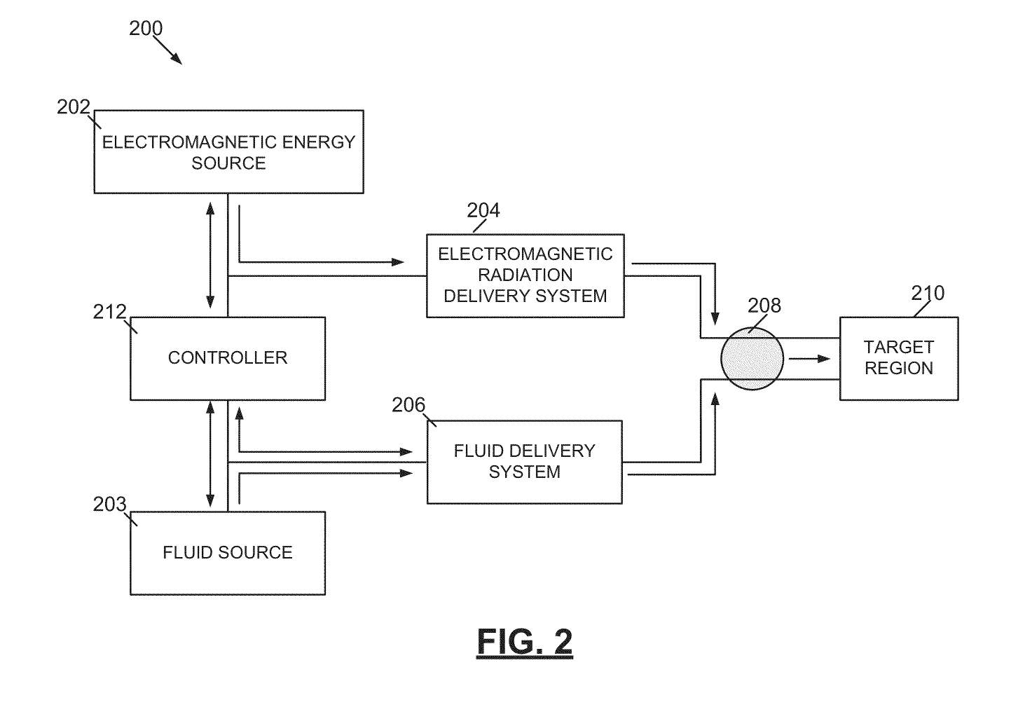

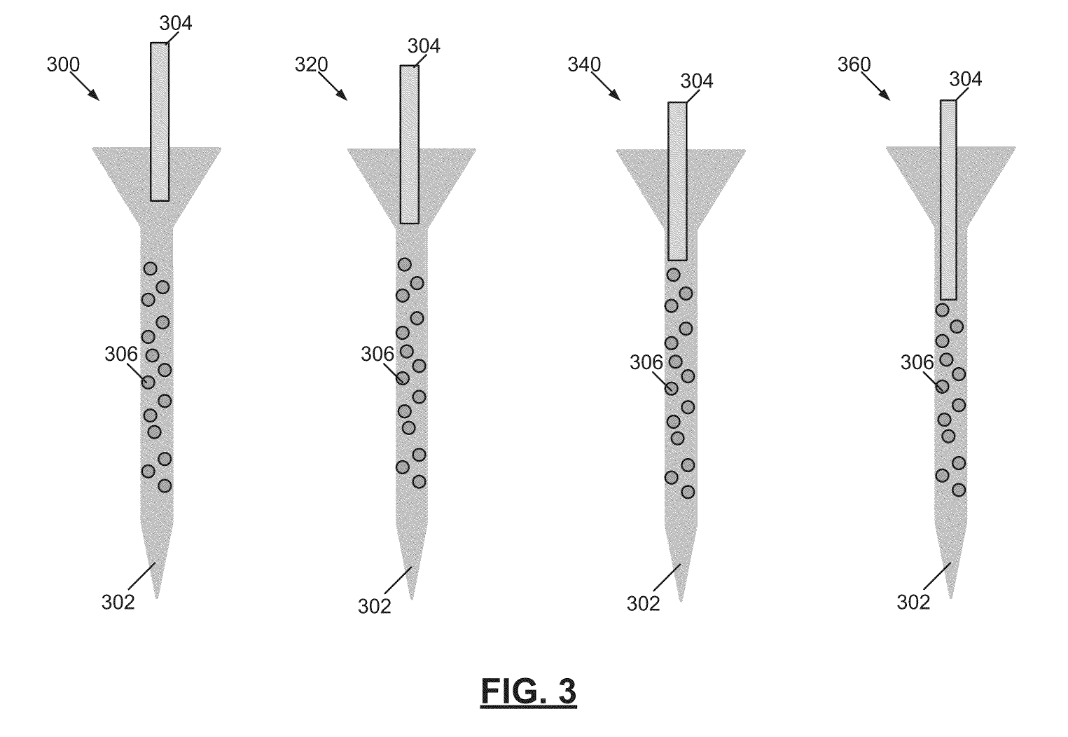

FIG. 2 depicts a block diagram of an example system 200 utilizing a dual-wavelength electromagnetic energy source 202 and a multi-mode fiber optic cable 204 to deliver a substance to a target region 210 in a vapor form. In the system 200 of FIG. 2, the electromagnetic energy source 202 includes sources 202A and 202B, which are configured to generate first and second wavelengths λ₁ and λ₂, respectively. With reference to FIGS. 1B and 1C, the first wavelength λ₁ is used to create the vapor bubble 142 within the fluid 104, and the second wavelength λ₂ is used to release the substance 108 in vapor form 182 into the vapor bubble 142. The electromagnetic energy source 202 is connected to both the multi-mode fiber optic cable 204 and a controller 212. The multi-mode fiber optic cable 204 routes the electromagnetic energy generated by the first and second sources 202A, 202B to a fiber optic tip 201. The fiber optic tip 201 is connected to an interaction zone 208 (e.g., positioned within the interaction zone 208) and delivers electromagnetic radiation to the interaction zone 208. The interaction zone 208 is a volume of space that extends into the target region 210 or that is adjacent to the target region 210. Further, with reference to FIGS. 1B and 1C, the interaction zone 208 includes an area in which electromagnetic radiation emitted from the fiber optic tip 106 and the fluid 104 interact to form the vapor bubble 142.

The interaction zone 208 is also connected to a fluid delivery system 206, which is configured to supply a fluid to the interaction zone 208. The fluid delivery system 206 receives the fluid from a fluid source 203. In one example, the fluid delivery system 206 is configured to fill the volume comprising the interaction zone 208 with the fluid. The interaction zone 208 may be a portion of a cavity, opening, canal, or passage, and the fluid delivery system 206 may be configured to fill the portion of the cavity, opening, canal, or passage with the fluid. In another example, the fluid delivery system 206 is an atomizer used to deliver atomized fluid particles into the interaction zone 208. In this example, the fluid is supplied as a stream or mist of conditioned fluid particles and may not completely fill the volume of the interaction zone 208. Further, the controller 212 to which the fluid delivery system 206 is connected may allow a user to specify a size and/or other characteristics of the fluid particles to be supplied to the interaction zone 208.

The fiber optic tip 201 is coated with the substance to be delivered to the target region 210. The substance is transparent to the first wavelength λ₁ supplied by the first source 202A and substantially absorbs light at the second wavelength λ₂ supplied by the second source 202B. In the interaction zone 208, a vapor bubble is created by exposing the fluid delivered by the fluid delivery system 206 to electromagnetic radiation at the first wavelength λ₁. The electromagnetic radiation at the first wavelength λ₁ is emitted by the fiber optic tip 201 and is substantially absorbed by the fluid in the interaction zone 208. During creation of the vapor bubble, the substance to be delivered to the target region 210 is released in vapor form into the vapor bubble by exposing the substance to electromagnetic radiation at the second wavelength λ2. The electromagnetic radiation at the second wavelength λ₂ is emitted by the fiber optic tip 201, which causes it to interact with the substance that coats the fiber optic tip 201. During this interaction, the electromagnetic radiation at the second wavelength λ₂ is substantially absorbed by the substance, causing it to vaporize into the vapor bubble that is being created.

The controller 212 is connected to the electromagnetic energy source 202, the fluid source 203, and the fluid delivery system 206, and is used to synchronize the delivery of the electromagnetic radiation and the fluid to the interaction zone 208. Additionally, the controller 212 controls various operating parameters of the electromagnetic energy source 202, the fluid source 203, and the fluid delivery system 206. For example, the controller 212 may be used to control the conditioning of the fluid from the fluid delivery system 206 (e.g., to control whether the fluid is delivered to the interaction zone 208 as a continuous volume of liquid or whether the fluid is atomized into discrete fluid particles). In another example, the electromagnetic energy source 202 includes one or more variable wavelength light sources, and the controller 212 allows a user to control the one or more variable wavelength light sources to change the first and/or second wavelengths λ₁, λ₂ emitted by the sources 202A, 202B. The user may change the first or second wavelengths λ₁, λ₂ emitted by the fiber optic tip 201 in order to tailor the emitted wavelengths to the absorption properties of different fluids and/or substances. In yet another example, the electromagnetic energy source 202 includes more than two sources of light. A larger number of sources may be used, such that the system 200 is equipped to work with a larger variety of fluids and/or substances. In such a system, the controller 212 may be used to select which of the multiple sources are used.

The electromagnetic energy source 202 may include a variety of different lasers, laser diodes, and/or other sources of light. The first and/or second sources 202A, 202B may be erbium, chromium, yttrium, scandium, gallium garnet (Er, Cr:YSGG) solid state lasers, which generate light having a wavelength in a range of 2.70 to 2.80 μm. Laser systems used in other examples include an erbium, yttrium, aluminum garnet (Er:YAG) solid state laser, which generates light having a wavelength of 2.94 μm; a chromium, thulium, erbium, yttrium, aluminum garnet (CTE:YAG) solid state laser, which generates light having a wavelength of 2.69 μm; an erbium, yttrium orthoaluminate (Er:YAL03) solid state laser, which generates light having a wavelength in a range of 2.71 to 2.86 μm; a holmium, yttrium, aluminum garnet (Ho:YAG) solid state laser, which generates light having a wavelength of 2.10 μm; a quadrupled neodymium, yttrium, aluminum garnet (quadrupled Nd:YAG) solid state laser, which generates light having a wavelength of 266 nm; an excimer laser, which generates light having a wavelength in a range of approximately 193 nm to 308 nm; and a carbon dioxide (CO2) laser, which generates light having a wavelength in a range of 9.0 to 10.6 μm.

FIG. 3 depicts example timing diagrams 300, 340, 380 illustrating aspects of a method for delivering a substance to a target region in a vapor form. Timing diagram 300 is a graph with the X axis representing units of time 304 and the Y axis representing peak power of emitted radiation at a first wavelength 302 in watts. With reference to FIG. 1B, the timing diagram 300 illustrates aspects relating to the delivery of the electromagnetic radiation at the first wavelength 144, which is used to create the vapor bubble 142 in the fluid 104. At a time of 1 ms, a pulse 306 of the electromagnetic radiation at the first wavelength is emitted by the fiber optic tip. The pulse 306 is highly absorbed by a fluid (e.g., the fluid 104 in FIG. 1B) and enables a vapor bubble to form in the fluid. In the timing diagram 300 of FIG. 3, the pulse 306 has a width of 50 μs, a pulse energy of 20 mJ, and a peak power of 400 W. FIG. 3 also depicts a second pulse 308 of the electromagnetic radiation at the first wavelength at a time of 101 ms, indicating that pulses of the electromagnetic radiation at the first wavelength are configured to be output at a frequency of 10 Hz (i.e., causing a period of 100 ms between pulses).

Timing diagram 340 is a graph with the X axis representing units of time 344 and the Y axis representing a diameter of a vapor bubble 342 in millimeters. With reference to FIG. 1B, the timing diagram 340 illustrates aspects of a bubble cycle of the vapor bubble 142 formed after the fluid 104 is excited by the electromagnetic radiation at the first wavelength 144. At a time of 1 ms, in response to the pulse 306 used to excite the fluid, a vapor bubble 346 is created in the fluid. In the timing diagram 340 of FIG. 3, the vapor bubble 346 has a peak diameter of 1 mm and a bubble cycle of nearly 1 ms. As illustrated in the graph 340, upon being exposed to the electromagnetic radiation at the first wavelength by the pulse 306, the fluid begins to form the vapor bubble 346. The vapor bubble 346 increases in diameter, reaches a maximum diameter, and finally collapses over the course of the nearly 1 ms bubble cycle. A second bubble 348 is formed in the fluid as a result of the second pulse 308 and has similar characteristics of the first bubble 346.

Timing diagram 380 is a graph with the X axis representing units of time 384 and the Y axis representing peak power of emitted radiation at a second wavelength 382 in watts. With reference to FIG. 1C, the timing diagram 380 illustrates aspects of the delivery of the electromagnetic radiation at the second wavelength 184 to the substance 108, which is used to release the substance 108 in vapor form 182 into the vapor bubble 142. At a time of 1 ms, a pulse 386 of the electromagnetic radiation at the second wavelength is emitted by the fiber optic tip. In the timing diagram 380 of FIG. 3, the pulse 386 has a width of nearly 1 ms, a pulse energy of 1 mJ, and a peak power of 1 W. The pulse 386 is launched at approximately the same time as the pulse 306, such that the substance to be delivered to the target region is released in vapor form into the vapor bubble 346 during the period of time that the vapor bubble 346 is being created. As illustrated in FIG. 3, the duration of the pulse 386 used to release the substance in vapor form into the vapor bubble 346 is substantially longer than the duration of the pulse 306 used to create the vapor bubble. Further, the peak power of the pulse 306 used to create the vapor bubble is substantially larger than the peak power of the pulse 386 used to release the substance in vapor form into the vapor bubble 346. A second pulse 388 of the electromagnetic radiation at the second wavelength is launched at a time of 101 ms to release the substance in vapor form into the vapor bubble 348.

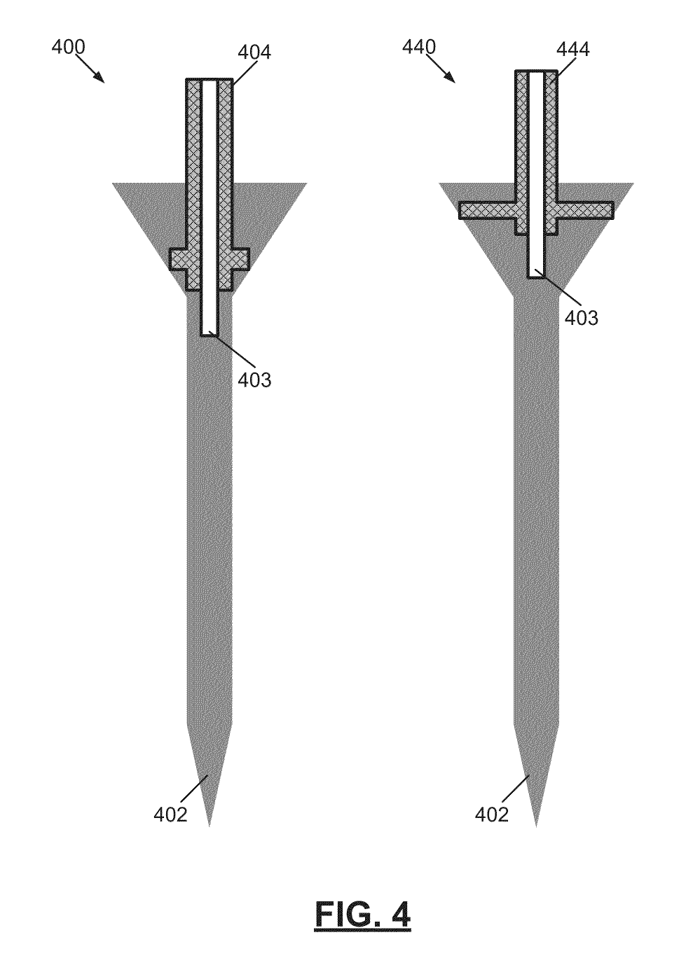

FIG. 4 depicts fiber optic cables 402 inserted into root canals 404 of a tooth 406 for intra-canal disinfection, cleaning, and/or medication delivery. The fiber optic cables 402 route electromagnetic radiation from an electromagnetic energy source 408 to fiber optic tips of the cables 402, which extend a substantial distance into the canals 404. The fiber optic cables 402 may be used with the systems and methods described in the preceding figures to deliver a substance to target regions of the tooth 406. In FIG. 4, the target regions to which the substance is to be delivered include various regions within the length of the canals 404. The substance to be delivered may include a medicine, cleaning agent, biologically-active particle, antiseptic, and/or antibiotic that is configured to clean the target regions, remove or kill bacteria within the target regions, disinfect the target regions, and/or apply a medical treatment to the target regions. In one example, the substance is iodine, and the iodine is delivered to the target regions of the root canals 404 in vapor form via a vapor bubble. In other examples, the fiber optic cables 402 may be inserted into a tooth cavity or other cavity, opening, or passage of a human body. Such cavities, openings, and passages may have dimensions on the order of the size of the fiber optic cable.

Properties of the fiber optic cables 402 and their associated fiber optic tips may be varied to accomplish the cleaning, disinfecting, and/or application of medical treatments to the target regions. For example, the fibers 402 may include single fibers or multi-fiber bundles of various designs (e.g., radially-emitting tips, side-firing tips, forward-firing tips, beveled tips, conical tips, angled tips). Further, the diameter of the fiber optic cables 402 may be varied, and the cables may have a tapered design with the fiber diameter increasing or decreasing over the length of the cable.

The fiber optic tips of the fiber optic cables 402 may be positioned at various distances from a target region to which the substance is to be delivered. In certain examples, the fiber optic tips of the fiber optic cables 402 are positioned a number of millimeters from the target region (e.g., positioned a number of millimeters away from the bottom of a canal, where the bottom of the canal is the target region), and in other examples, the fiber optic tips may be positioned directly in contact with the target region (i.e., adjacent to the target region). Further, the fiber optic tips of the fiber optic cables 402 may not be inserted into the canals 404 but may instead be may be centered above the canal, near the entrance to the canal.

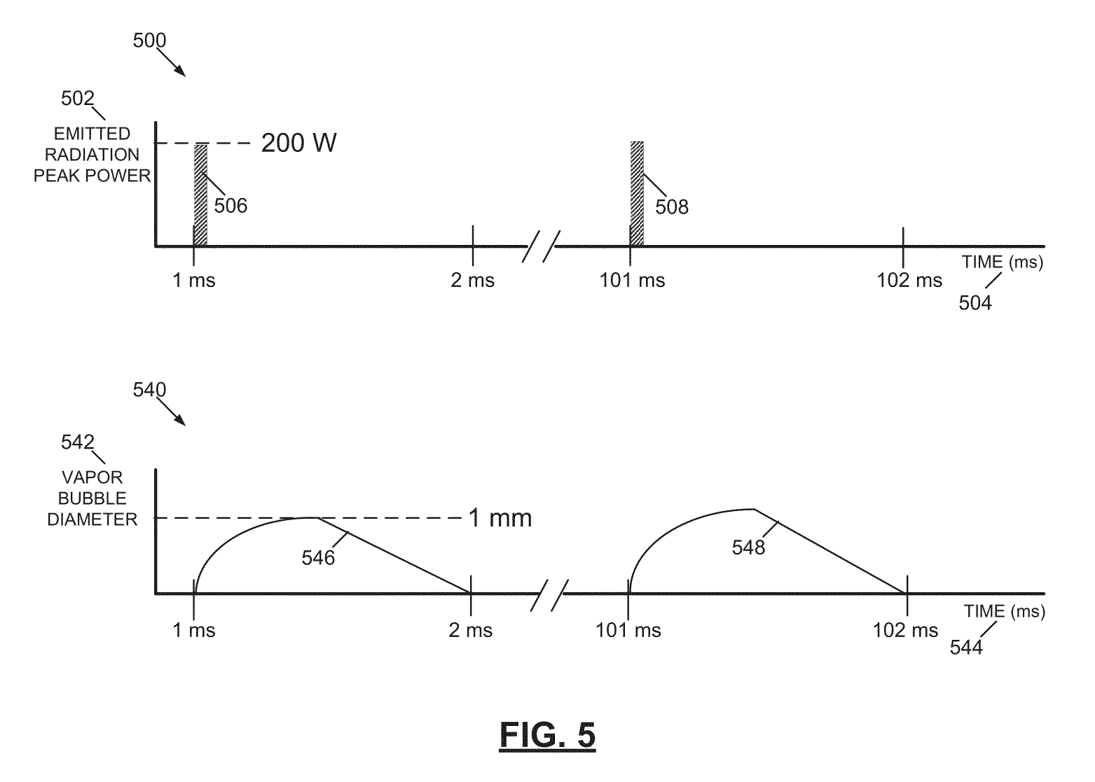

FIG. 5 illustrates an example system 500 for delivering a medication or cleaning agent 508 to a target area 502 via a plurality of vapor bubbles 510 carrying the medication or the cleaning agent 508 in a vapor form. In FIG. 5, a fluid 504 is placed in the target region 502. As in FIGS. 1A, 1B, and 1C, the target region 502 is a cavity, canal, opening, or surface to which it is desired that the medication or cleaning agent 508 be delivered. The target region 502 is of a small size, on the order of a size of a fiber optic tip 506, and may be a cavity, canal, opening, or surface of the human body. In addition to the fluid 504 being placed in the target region 502, the fiber optic tip 506 is also positioned within the target region 502 or adjacent to the target region 502. The fiber optic tip 506 is used to emit electromagnetic radiation and is connected via a multi-mode fiber optic cable to an electromagnetic energy source, which generates electromagnetic radiation at first and second wavelengths 503, 505. The fiber optic tip 506 is coated in the substance 508, such that the electromagnetic radiation 503, 505 emitted by the tip 506 interacts with the substance 508 as it is emitted from the tip 506.

In the example of FIG. 5, a vapor bubble 510 is created by exposing the fluid 504 to the electromagnetic radiation at the first wavelength 503. The first wavelength 503 is configured to be substantially absorbed by the fluid 504 and transparent to the substance 508. Due to the absorption of the radiation at the first wavelength 503 in the fluid 504, the vapor bubble 510 is created in the fluid 504. The substance 508 is released in a vapor form into the vapor bubble 510 by exposing the substance 508 to the electromagnetic radiation at the second wavelength 505. The second wavelength 505 is substantially absorbed by the substance 508, causing the substance 508 to evaporate into the vapor bubble 510 as it is being formed. The electromagnetic radiation at the first and second wavelengths 503, 505 are delivered as light pulses to the fluid 504 and the substance 508, respectively, and the light pulses of the two wavelengths are launched at substantially similar times (e.g., as illustrated in FIGS. 3A and 3C).

As illustrated in FIG. 5, a plurality of vapor bubbles 510 containing the substance 508 in vapor form may be created. In one example, the plurality of bubbles is created by exposing the fluid 504 to a plurality of light pulses of the first wavelength 503 and exposing the substance 508 to a plurality of light pulses of the second wavelength 505. Repetitive exposures of the fluid 504 and the substance 508 create a “bubbling” fluid, where each bubble 510 contains the substance 508 in vapor form. Adjusting parameters of the laser radiation at the first and second wavelengths 503, 505 alters characteristics of the bubbling effect (e.g., volume of bubbles, rate of bubble production, speed of release of the substance 508). In another example, the vapor bubbles 510 are created by pulsing the electromagnetic radiation at the first wavelength 503 and allowing the substance 508 to be exposed to electromagnetic radiation at the second wavelength 505 via a steady state exposure, rather than exposure via pulses.

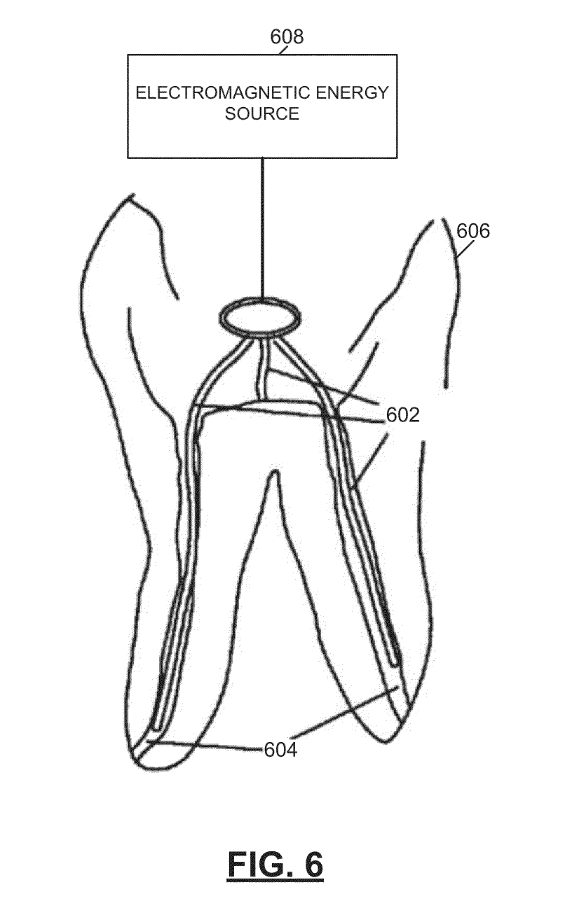

Although the systems described in the preceding figures utilize multiple wavelengths of light to achieve the creation of bubbles and the filling of the bubbles with the substance (e.g., first and second wavelengths 503, 505 of FIG. 5), in other examples, only a single wavelength of light is used. FIGS. 6A and 6B depict example systems 600, 640 that utilize a spraying technique to disperse medication 603 into a vapor bubble 608 for delivery to a target region 602. As in example systems previously described (e.g., the system of FIGS. 1A, 1B, and 1C), a fluid 604 and a fiber optic tip 606 are positioned within the target region 602. The fiber optic tip 606 is configured to emit electromagnetic radiation at a wavelength 601 that is generated by an electromagnetic energy source. The vapor bubble 608 is created within the target region 602 by exposing the fluid 604 to the electromagnetic radiation at the wavelength 601 via the fiber optic tip 606, as in example systems previously described.

In contrast to the systems previously described, in the example systems 600, 640 of FIGS. 6A and 6B, the fiber optic tip 606 is not coated with the medication 603 to be delivered to the target region 602. Further, the medication 603 to be delivered to the target region 602 is not dispersed within the vapor bubble 608 by exposing the medication 603 to electromagnetic radiation at a second wavelength. Rather, as illustrated in FIGS. 6A and 6B, the medication 603 is placed in the vapor bubble 608 via a spraying technique. In FIG. 6A, an apparatus 605 is used to store the medication 603 and to spray the medication 603 into the vapor bubble 608 for delivery to the target region 602. The apparatus 605 is attached to the fiber optic tip 606. Similarly, an apparatus 645 in FIG. 6B is used to store the medication 603 and to spray the medication 603 into the vapor bubble 608. The apparatus 645 of FIG. 6B is separate from the fiber optic tip 606. In the systems 600, 640, the medication 603 may be released into the vapor bubble 608 in a solid, liquid, and/or gaseous form. In other example systems, the medication 603 is not sprayed into the vapor bubble 608 but is rather released via a different non-explosive process that does not involve irradiation of the medication 603 at a second wavelength of light (e.g., thermal, mechanical, or electrical means to release the medication 603 into the vapor bubble 608).

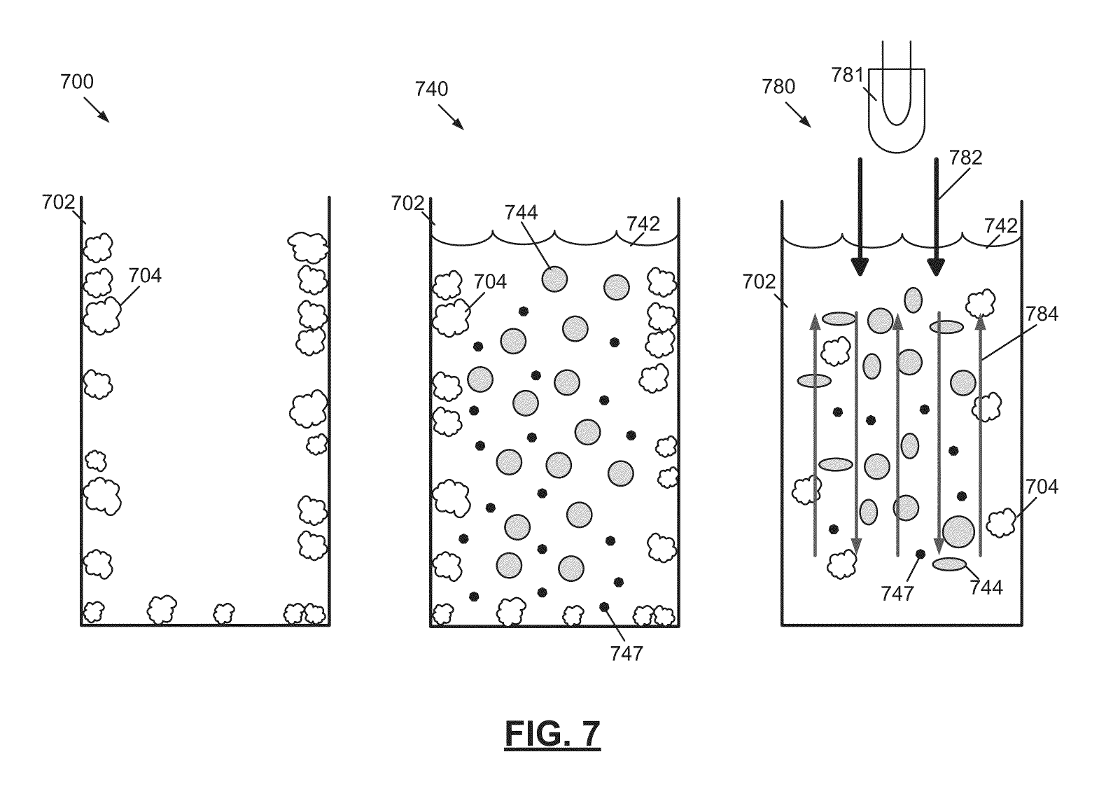

FIG. 7 depicts a block diagram of an example system utilizing an electromagnetic energy source 702 with a plurality of laser sources 703 to deliver a medicine to a target region 710 in a vapor form. In the system 700 of FIG. 7, the electromagnetic energy source 702 includes n separate electromagnetic energy sources 703 (e.g., lasers, laser diodes) configured to produce electromagnetic radiation at wavelengths λ₁, λ₂, λ₃, λ₄, . . . λₙ. The n electromagnetic energy sources are utilized to enable a variety of different fluids and medicines 705 to be used with the system 700. As noted previously, forming a vapor bubble and releasing medicine into the vapor bubble may require that the fluid and the medicine be matched with particular light emitting sources (i.e., the fluid and the medicine must have high absorption properties at the wavelengths of light of the particular light emitting sources). Thus, by including the n electromagnetic energy sources 703, a wider variety of fluids and/or medicines may be used with the system 700. The n electromagnetic energy sources 703 may be used to expose the fluid to create the vapor bubble and/or expose the medicine 705 to be dispersed in the vapor bubble.

The electromagnetic energy source 702 is connected to both a multi-mode fiber optic cable 704 and a controller 712. The multi-mode fiber optic cable 704 routes the electromagnetic energy generated by the n sources 703 to a fiber optic tip 701. The fiber optic tip 701 may be coated with any of n different medicines 705 (e.g., various disinfectant solutions or medications used for injections). The fiber optic tip 701 is connected to an interaction zone 708 (e.g., positioned within the interaction zone 708) and delivers electromagnetic radiation to the interaction zone 708. The interaction zone 708 is a volume of space that extends into the target region 710 or that is adjacent to the target region 710. The interaction zone 708 is also connected to a fluid delivery system 706, which is configured to supply a fluid to the interaction zone 708.

The controller 712 is connected to both the electromagnetic energy source 702 and to the fluid delivery system 706, and is used to synchronize the delivery of the electromagnetic radiation and the fluid to the interaction zone 708. Additionally, the controller 712 includes a graphical user interface (GUI) that allows a user to control various operating parameters of the system 700. For example, the GUI allows the user to select the fluid and the medication 705 that are to be used with the system 700. Based on the selections, the controller 712 selects certain sources of the n light sources to be used (i.e., the controller 712 selects sources from the n light sources 703 that are best matched to the user's selected fluid and medication). The GUI of the controller 712 also includes a laser selector that allows the user to manually choose which of the n light sources 703 are to be used for exposing the fluid and dispersing the medicine 705 into the vapor bubble.

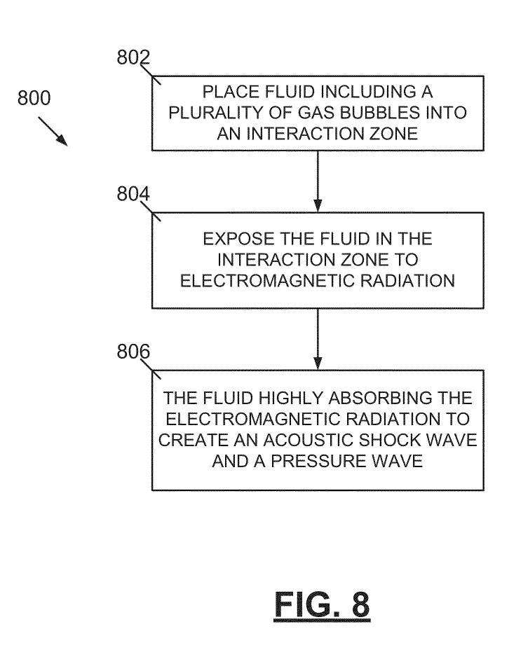

FIG. 8 is a flowchart 800 illustrating an example method for delivering a substance to a target region in a vapor form. At 802, a fluid is placed within an interaction zone. The interaction zone is a volume that extends into the target region or that is adjacent to the target region. At 804, an electromagnetic radiation emitting fiber optic tip is positioned within the interaction zone. The fiber optic tip contains the substance that is transparent to a first wavelength of energy and that substantially absorbs a second wavelength of energy. At 806, a vapor bubble is created within the interaction zone by exposing the fluid to electromagnetic radiation at the first wavelength. The electromagnetic radiation at the first wavelength is substantially absorbed by the fluid in the interaction zone. At 808, the substance is released in a vapor form into the vapor bubble by exposing the substance to electromagnetic radiation at the second wavelength. The electromagnetic radiation at the first and second wavelengths is emitted by the fiber optic tip.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Further, as used in the description herein and throughout the claims that follow, the meaning of “each” does not require “each and every” unless the context clearly dictates otherwise. Finally, as used in the description herein and throughout the claims that follow, the meanings of “and” and “or” include both the conjunctive and disjunctive and may be used interchangeably unless the context expressly dictates otherwise; the phrase “exclusive of” may be used to indicate situations where only the disjunctive meaning may apply.

It is claimed:

1. A method for delivering a substance to a target region in a vapor form, the method comprising:
placing a fluid within an interaction zone;
positioning an electromagnetic radiation emitting fiber optic tip within the interaction zone, the fiber optic tip containing the substance that is transparent to a first wavelength of energy and that substantially absorbs a second wavelength of energy;
creating a vapor bubble within the interaction zone by exposing the fluid to electromagnetic radiation at the first wavelength, the electromagnetic radiation at the first wavelength being substantially absorbed by the fluid in the interaction zone;
releasing the substance in a vapor form into the vapor bubble by exposing the substance to electromagnetic radiation at the second wavelength, the electromagnetic radiation at the first and second wavelengths being emitted by the fiber optic tip; and
using the vapor bubble to deliver the substance to the target region in the vapor form.

2. The method of claim 1, wherein the interaction zone is a volume that extends into to the target region or that is adjacent to the target region.

3. The method of claim 1, wherein the method for delivering the substance to the target region is applied in a medical or dental procedure.

4. The method of claim 1, wherein the substance is a medicine, cleaning agent, biologically-active particle, antiseptic, or antibiotic that is configured to clean the target region, remove or kill bacteria in the target region, disinfect the target region, or apply a medical treatment to the target region.

5. The method of claim 1, wherein the substance is iodine, and wherein the iodine is configured to kill bacteria within the fluid or on walls of the target region.

6. The method of claim 1, wherein the target region is a cavity, opening, or passage having dimensions similar in size to dimensions of the fiber optic tip.

7. The method of claim 6, wherein the fiber optic tip is positioned within the interaction zone by inserting the fiber optic tip into the cavity, opening, or passage or placing the fiber optic tip near an entrance of the cavity, opening, or passage.

8. The method of claim 1, wherein the target region is a root canal passage, tubule of a tooth, tooth cavity, tooth surface, or blood vessel.

9. The method of claim 1, wherein the electromagnetic radiation at the first and the second wavelengths are generated by an electromagnetic energy source, and wherein the electromagnetic energy source includes first and second light emitting sources configured to create the electromagnetic radiation at the first and the second wavelengths, respectively.

10. The method of claim 9, further comprising:
changing the first wavelength or the second wavelength emitted by the fiber optic tip via the electromagnetic energy source, wherein the electromagnetic energy source is a variable wavelength light source.

11. The method of claim 9, wherein the radiation at the first and the second wavelengths are routed from the electromagnetic energy source to the fiber optic tip via a multi-mode fiber optic cable.

12. The method of claim 1, wherein the fluid is water-based, and wherein the first wavelength is within the range of 2.6 μm to 3.1 μm.

13. The method of claim 12, wherein the second wavelength is within the ultraviolet, visible, or near-infrared regions of the electromagnetic spectrum.

14. The method of claim 1, wherein the fluid is exposed to the electromagnetic radiation at the first wavelength via a first light pulse emitted by the fiber optic tip, and wherein the substance is exposed to the electromagnetic radiation at the second wavelength via a second light pulse emitted by the fiber optic tip.

15. The method of claim 14, wherein a duration of the second light pulse is substantially longer than a duration of the first light pulse.

16. The method of claim 14, wherein a peak power of the first light pulse is substantially larger than a peak power of the second light pulse.

17. The method of claim 14, wherein the substance is released in the vapor form into the vapor bubble during a period of time that the vapor bubble is being created, and wherein the first and second light pulses are launched at similar times.

18. The method of claim 17, wherein the period of time that the vapor bubble is being created is on the order of 1 millisecond.

19. The method of claim 1, comprising:
creating a plurality of vapor bubbles within the interaction zone by exposing the fluid to a plurality of light pulses at the first wavelength; and
releasing the substance in the vapor form into the plurality of vapor bubbles by exposing the substance to the electromagnetic radiation at the second wavelength, the electromagnetic radiation at the second wavelength including a plurality of light pulses at the second wavelength or a steady-state exposure of the substance at the second wavelength.

20. A system for delivering a substance to a target region in a vapor form, the system comprising:
a fluid, the fluid being located within an interaction zone;
an electromagnetic radiation emitting fiber optic tip, the fiber optic tip being positioned within the interaction zone and containing the substance that is transparent to a first wavelength of energy and that substantially absorbs a second wavelength of energy;
an electromagnetic energy source configured to generate electromagnetic radiation at the first and second wavelengths for emission by the fiber optic tip, the emitted electromagnetic radiation at the first wavelength being substantially absorbed by the fluid and being configured to create a vapor bubble within the fluid, and the emitted electromagnetic radiation at the second wavelength being configured to release the substance in a vapor form into the vapor bubble.

21. A method for delivering a substance to a target region, the method comprising:
placing a fluid within an interaction zone, the interaction zone being a volume that extends into the target region or that is adjacent to the target region;
positioning an electromagnetic radiation emitting element within the interaction zone, the element containing the substance that is transparent to a particular wavelength of energy;
creating a vapor bubble within the fluid by exposing the fluid to electromagnetic radiation at the particular wavelength, the electromagnetic radiation at the particular wavelength being emitted by the electromagnetic radiation emitting element and being substantially absorbed by the fluid in the interaction zone; and
during the creation of the vapor bubble, releasing the substance into the vapor bubble.
